(19) United States
(12) Patent Application Publication
(10) Pub. No.: US 2002/0168347 A1
(43) Pub. Date: Nov. 14, 2002

(54) USE OF MONOCYTES DERIVED CELLS, ANTIGENS AND ANTIBODIES FOR OPTIMAL INDUCTION OF IMMUNOTHERAPEUTIC EFFICIENCY

(76) Inventors: JACQUES BARTHOLEYNS, BURES-SUR-YVETTE (FR); YVES FOURON, MARLBOROUGH, MA (US)

Correspondence Address: BIERMAN MUSERLIAN AND LUCAS, 600 THIRD AVENUE, NEW YORK, NY, 10016

(21) Appl. No.: 09/080,698

(22) Filed: May 18, 1998

(30) Foreign Application Priority Data
Apr. 9, 1998 (EP) 984008839

(51) Int. Cl. A61K 48/00; A01N 63/00

(52) U.S. Cl. 424/93.21

(57) ABSTRACT

The invention relates to monocyte derived cells, in a purified form and substantially free of contaminants, presenting antigenic epitopes on their membranes after interiorization and processing of at least an antigen-antibody complex formed between an antigen and an antibody, under appropriate conditions, said epitopes corresponding to proteolytic degradation products of said antigen,

with said antibody being directed against a tumor or against an infectious agent, and

with said antigen being fragments of tumor or of infectious agent, including membranes or tumor apoptotic bodies, or purified tumor or infectious antigens or being recombinant tumor or infectious antigens.

The invention relates to the use of monocytes derived cells, antigens and antibodies for optimal induction of immunotherapeutic efficiency.

Macrophages play a major role in the antitumoral response, and they are able to be activated by immunological activators against cancer cells (Adams D. and Hamilton T.: “Activation of macrophages for tumor cell kill: effector mechanism and regulation”; in Heppner & Fulton (eds), Macrophages and cancer. CRC Press, 1988, p. 27; Fidler M. Macrophages and metastases. A biological approach to cancer therapy. Cancer Res. 45: 4714, 1985).

Furthermore, macrophages, or other cells derived from monocytes or from their precursors, with their strong capacity for endocytosis, digestion, and surface antigen presentation, are capable of inducing a specific immune response.

Macrophages represent the first natural line of defense against infectious agents (bacteria, virus) which are normally killed. However, resistant pathogens can develop ways to escape recognition and killing by macrophages. Protection cannot be very effectively achieved with the attenuated infectious agents or with their recombinant peptidic components.

Monocytes derived cells (MDCs) are immune cells such as obtained by culture of blood mononuclear cells in non adherent gas permeable plastic or Teflon bags for 5 to 10 days at 37° C., in O₂/CO₂ atmosphere. Their culture medium (RPMI, IMDM, AIM5 (Gibco) or X-VIVO (Biowhittaker)) contains eventually cytokines or ligands as defined in patents n° PCT/EP93/01232, n° WO94/26875 or EP 97/02703 or in the articles mentioned below:

“Autologous lymphocytes prevent the death of monocytes in culture and promote, as do GM-CSF, IL-3 and M-CSF, their differentiation into macrophages”. (Lopez M., Martinache Ch., Canepa S., Chokri M., Scotto F., Bartholeyns J.; J. of Immunological Methods, 159: 29-38, 1993);

“Immune therapy with macrophages: Present status and critical requirements for implementation” (Bartholeyns J., Romet-Lemonne J -L., Chokri M., Lopez M.; Immunobiol., 195: 550-562, 1996);

“In vitro generation of CD83⁺ human blood dendritic cells for active tumor immunotherapy” (Thurnher M., Papesh C., Ramoner R., Gastlt G. and al.; Experimental Hematology, 25: 232-237, 1997);

“Dendritic cells as adjuvants for immune-mediated resistance to tumors” (Schuler G. and Steinman R. M.; J. Exp. Med., 186: 1183-1187, 1997).

All these patents applications and articles are included herein for references.

They can be activated by INF-γ at the end of culture to obtain in particular cytotoxic macrophages. They can be centrifuged to be concentrated and purified before resuspension in isotonic solution.

Monocytes derived cells (MDCs) can either be killer macrophages, phagocytozing cells, growth factors and cytokines releasing cells, or dendritic cells according to their conditions of differentiation. Dendritic cells can for example be obtained as described in “In vitro generation of CD83⁺ human blood dendritic cells for active tumor immunotherapy” (Thurnher M., Papesh C., Ramoner R., Gastlt G. and al.; Experimental Hematology, 25: 232-237, 1997) and “Dendritic cells as adjuvants for immune-mediated resistance to tumors” (Schuler G. and Steinman R. M.; J. Exp. Med., 186: 1183-1187, 1997), and EP n° 97/02703.

Mature dendritic cells are very potent antigen presenting cells to initiate an immune response. The dendritic cells can be characterized by the induction of T cell proliferation and by their phenotype (presence of CD80, CD86, CD83, MHC-I, MHC-II on their membranes).

Human IgM or IgG antitumoral antibodies are developed to target tumors or metastases. Clinical anti-cancer responses achieved with these monoclonal human antibodies are not impressive due to unsolved problems of tissue distribution and disseminated sites, limited access to the tumor or to low local concentration, inadequate pharmacokinetics or ineffective dosage, and mainly to the lack of effector cells impairing cellular and humoral immune responses against the relevant tumor.

IgG, IgA and IgM antibodies directed against infectious agents can be isolated in human plasma an are prepared as monoclonal antibodies. These antibodies do very seldom neutralize the infection as such; a cellular immune response is required. Therapeutic vaccines are therefore difficult to develop with attenuated infectious agents or with their recombinant peptidic components.

Pan carcinomic tumor antigens (present in breast, prostate, lung, melanoma, glioma, neuroblastoma and corectal tumors for example) are described and can be targeted, at least in vitro, by specific human monoclonal antibodies. These pan antitumor antibodies do not react with normal cells and tissues such as epithelium, fibroblasts, neuroectodermal or muscle cells. However, these antibodies bind through their Fc part to reticuloendothelial cells such as macrophages bearing Fc receptors. Therefore most antibodies do not effectively reach the tumor target antigen.

The inefficiency of anti-tumor or anti-infection antibodies is overcome by the present invention which enables to enhance cellular and humoral anti-tumor and antiinfection immune response by initiating ex vivo and controlling the interaction between target antigens, specific antibodies and effector monocyte derived cells. One of the aim of the invention is to provide monocytes derived cells presenting antigenic epitopes on their membrane.

Another aim of the invention is to provide a process for preparing monocytes derived cells presenting antigenic epitopes on their membrane.

Another aim of the invention is to provide a method for the treatment or prevention of cancer.

Another aim of the invention is to provide a method for the treatment or prevention of viral or bacterial infections.

The invention relates in a general embodiment to monocytes derived cells, in a purified form and substantially free of contaminants, presenting antigenic epitopes on their membranes after interiorization and processing of at least an antigen-antibody complex formed between an antigen and an antibody, under appropriate conditions, said epitopes corresponding to proteolytic degradation products of said antigen,

with said antibody being directed against a tumor or against an infectious agent, and

with said antigen being fragments of tumor or of infectious agent, including membranes or tumor apoptotic bodies, or infectious antigens or being recombinant tumor or infectious antigens.

The present invention describes an ex-vivo method to favor and control the interaction between autologous macrophages or monocytes derived antigen presenting cells, antibodies such as pan anti-tumor antibodies and relevant tumor antigen(s). This controlled interaction (e.a. tumor Ag-pan anti-tumor Ab-effector macrophage) allows a synergistic enhancement of the relevant biological activities and as a result induces in vivo cellular and humoral anti-tumor or anti-infection immune response. Tumor apoptotic bodies are particularly adequate as source of tumor antigen.

The invention takes advantage of an antigen, particularly the availability of the pan tumor antigen, or the tumor apoptotic body as source of tumor antigen, and of the corresponding specific human antibody, to create in vitro a molecular complex which is readily internalized by monocytes derived macrophages or antigen presenting cells. The endocytosis of the immune complex mainly via Fc receptors of the macrophages allows effective processing in vitro. The invention enables also the favored ex vivo interaction between fragments of a patient's tumor membrane, IgM monoclonal antibodies recognizing tumor epitopes on these membranes, and autologous macrophages, resulting in adequate processing of the tumor.

In the case of cancer, by way of example, the immune response is forced in the present invention by initial in vitro interaction between the complex pan tumoral Ag-antitumoral Ab-autologous monocytes derived antigen presenting cells or macrophages. This interaction allows endocytosis of the carcinoma antigen or of the apoptotic body via Fc receptors, adequate processing, interaction with MHCI and MHCII molecules and presentation of antigenic tumor peptides on the cell membrane. The tumor epitopes presented on the membrane of the effector cells are recognized by autologous T lymphocytes of the patient. The pan tumoral nature of the antigen avoids the MHC restriction which limits the use of purified tumor antigens/epitopes to a few selected patients. The injection of this preparation to patients with most types of carcinomas induces potent specific humoral and cellular immune responses against the relevant tumor.

In the case of infection, an alternative approach to induce effective immunotherapy is to inject a ternary complex formed between the antibody, the infectious agent and macrophages processing the complex, presenting adequately epitopes of the pathogen and forcing the induction of a long lasting immunity against the pathogen and against subsequent infections.

The expression “purifed form” means that the ternary complex formed by tumor or infectious antigens, antitumor or anti-infectious IgM, IgA, IgG antibodies, and effector macrophages or monocyte derived cells is segregated from other cell types or complexes.

The expression “tumor apoptotic bodies” refers to microbodies isolated from tumor cells or cell lines induced to apoptosis, containing mitochondria and DNA within a membrane exposing tumor antigens (Bellone M., Iezzi G., Roverel Galati G. et al.—“Processing of engulfed apoptotic bodies yields T cell epitopes”, Journal of Immunology, 159: 5391-5399, 1997).

The expression “substantially free of contaminants” means that macrophages or monocyte derived cells are 90% pure and that no contaminants other than the antibody and the tumor Ag or apoptotic bodies or infectious antigens are present.

The expression “antigen-antibody complex formed between an antigen and an antibody under appropriated conditions” means that antigen and corresponding antibody associate by high affinity (10⁴ to 10¹¹ L/M) non covalent binding, the antigen can be particular as in the case of apoptotic bodies, increasing the avidity of the complex formed.

The expression “epitopes corresponding to proteolytic degradation products of said antigen” means that the antigen phagocytosed in vacuoles of macrophages or monocyte derived cells is digested by proteases into smaller polypeptide fragments.

The monocyte derived cells of the invention can be identified by the following method : presence of Fcγ receptors (CD16, CD32, CD64), of Fcμ and Fcγ receptors (CD89) and of CR3, of CR5 and of mannose receptor on their membranes at the same time as high levels of MHCI and of MHCII molecules.

The invention also relates to a process for the preparation of monocyte derived cells presenting antigenic epitopes on their membranes after interiorization and processing of at least an antigen-antibody complex, formed between an antigen and an antibody, said process comprising the following steps:

a) preparation of the monocytes derived cells according to the following method:

1) recovery of blood derived mononuclear cells directly from blood apheresis or from blood bag collection, followed if necessary by centrifugation, to eliminate a substantial part of red blood cells granulocytes and platelets, and collection of peripheral blood leukocytes;

2) washing peripheral blood leukocytes obtained at the preceeding steps for instance by centrifugation (to remove 90% of platelets, red blood cells and debris) to obtain mononuclear cells;

3) resuspension of the total mononuclear cells obtained at the preceeding step in culture medium (RPMI or IMDM type) at 10⁶ to 2.10⁷ cells/ml, possibly completed by cytokines and/or autologous serum, and culture for 5 to 10 days at 37° C. under O₂/CO₂ atmosphere in hydrophobic gas permeable bags, to obtain monocyte derived cells and contaminating lymphocytes;

b) addition of antigens and antibodies to the monocyte derived cells obtained at the preceeding step to form a ternary complex between monocyte derived cells, an antigen and an antibody;

c) incubation of said ternary complex for a time and at a temperature sufficient to allow endocytosis into intracellular vacuoles of the monocyte derived cells and processing of the antigen-antibody complex, with said processing consisting of digestion of the antigen-antibody complex and association of the epitopes of the antigen resulting from the digestion with MHC molecules, to obtain monocyte derived cells presenting antigenic epitopes on their membranes.

The preparation of the monocyte derived cells can be carried out as described in patents n° PCT/EP93/01232, n° WO94/26875 or EP 97/02703 or in the articles mentioned below:

“Autologous lymphocytes prevent the death of monocytes in culture and promote, as do GM-CSF, IL-3 and M-CSF, their differentiation into macrophages”. (Lopez M., Martinache Ch., Canepa S., Chokri M., Scotto F., Bartholeyns J.; J. of Immunological Methods, 159: 29-38, 1993);

“Immune therapy with macrophages: Present status and critical requirements for implementation” (Bartholeyns J., Romet-Lemonne J -L., Chokri M., Lopez M.; Immunobiol., 195: 550-562, 1996);

“In vitro generation of CD83⁺ human blood dendritic cells for active tumor immunotherapy” (Thurnher M., Papesh C., Ramoner R., Gastlt G. and al.; Experimental Hematology, 25: 232-237, 1997);

“Dendritic cells as adjuvants for immune-mediated resistance to tumors” (Schuler G. and Steinman R. M.; J. Exp. Med., 186: 1183-1187, 1997).

According to an advantageous embodiment of the invention, the process is such that

said antibody is directed against a tumor or against an infectious agent,

with said antigen is fragments of tumor or of infectious agent, including membranes or tumor apoptotic bodies, or purified tumor or infectious antigens or is a recombinant tumor or infectious antigen.

According to an advantageous embodiment of the invention, in the step of addition, said antigens and antibodies are in the form of a complex.

According to an another embodiment of the invention, in the step of addition, said antigens and antibodies are not in the form of a complex.

According to an advantageous embodiment of the invention, the process comprises the additional following step:

d) centrifugation of the monocyte derived cells presenting antigenic epitopes on their membranes, washing and resuspension, for instance in isotonic medium, to obtain a suspension of the above defined monocyte derived cells.

According to an another advantageous embodiment of the invention, in the process above defined, the step of centrifugation is followed by

e) freezing at temperature below or equal to −80° C. aliquots of the above said suspension, with the addition of a cryopreservative.

According to an another advantageous embodiment of the process, the step of freezing is followed by

f) melting said above frozen aliquots at a temperature enabling to obtain a suspension of monocyte derived cells presenting antigenic epitopes on their membranes, for instance at 4° C., washing said suspension and resuspending it, for instance in an isotonic medium, to obtain a suspension of monocyte derived cells presenting antigenic epitopes on their membranes.

Advantageously, in the process of the invention, the antibodies are human or humanised IgG, IgA or preferably IgM (for induction of primary immune response) directed against tumor antigens or against infectious antigens (viral or bacterial).

Advantageously, in the process of the invention, the antigens are purified tumor, viral or bacterial antigens (polypeptides, glycopeptides, oligosacharide) or membrane fragments serving as complex antigens.

Advantageously, in the process of the invention, the antigens are pan tumor antigens present on different tumor types.

The invention also relates to a ternary complex in a purified form, and substantially free of contaminants, between monocytes derived cells, an antigen and an antibody

with said antibody being directed against a tumor or against an infectious agent,

with said antigen being fragments of tumor or of infectious agent including membranes or tumor apoptotic bodies, or purified tumor or infectious antigens or being recombinant tumor or infectious antigens, and

with said antigen and said antibody being liable to form an antigen-antibody complex under appropriate conditions.

The expresssion “ternary complex” designates a complex formed ex vivo between an antigen (tumor or infections origin), an antibody recognizing that antigen and a macrophage or monocyte derived cell binding the previous binary complex on its membrane before internalization.

A ternary complex can be identified by electron microscopy or by FACS analysis (fluorescence cell analysis) of the monocyte derived cell and of the molecules expressed on its membrane. High affinity binding between the antigen and the antibody occurs before binding of the complex to the monocyte derived cell membrane. Binding occurs at a temperature below 10° C. while internalization occurs only at a temperature above 20° C.

The expression “purified form” means that the ternary complex formed by tumor or infectious antigens, anti-tumor or anti-infectious IgM, IgA, IgG antibodies, and effector macrophages or monocyte derived cells is segregated from other cell type or complexes.

The expression “substantially free of contaminants” means that macrophages or monocyte derived cells are 90% pure and that no contaminants other than the antibody and the tumor or infectious antigens are present.

A process for the preparation of a ternary complex between monocyte derived cells, an antigen and an antibody as above defined, comprising the following steps:

1) recovery of blood derived mononuclear cells directly from blood apheresis or from blood bag collection, followed if necessary by centrifugation, to eliminate a substantial part of red blood cells granulocytes and platelets, and collection of peripheral blood leukocytes;

2) washing peripheral blood leukocytes obtained at the preceeding steps for instance by centrifugation (to remove 90% of platelets, red blood cells and debris) to obtain mononuclear cells;

3) resuspension of the total mononuclear cells obtained at the preceeding step in culture medium (RPMI or IMDM type) at 10⁶ to 2.10⁷ cells/ml, possibly completed by cytokines and/or autologous serum, and culture for 5 to 10 days at 37° C. under O₂/CO₂ atmosphere in hydrophobic gas permeable bags, to obtain monocyte derived cells and contaminating lymphocytes;

b) addition of antigens and antibodies to the monocyte derived cells obtained at the preceeding step to form of a ternary complex between monocyte derived cells, an antigen and an antibody.

In the process above defined, said antigens and antibodies are advantageously in the form of a complex.

In another embodiment of the process above defined, said antigens and antibodies are not in the form of a complex.

The invention also relates to monocyte derived cells presenting antigenic epitopes on their membranes such as obtained according to the process of the invention.

The invention also relates to a ternary complex between monocyte derived cells, an antigen and an antibody such as obtained according to the process above defined.

The invention also relates to a pharmaceutical composition containing as active substance monocyte derived cells presenting antigenic epitopes on their membranes according to the invention, in association with a pharmaceutically acceptable vehicle.

The invention also relates to a pharmaceutical composition containing as active substance a ternary complex according to the invention, in association with a pharmaceutically acceptable vehicle.

The pharmaceutical compositions of the invention are advantageously in the form of sterile injectable preparations.

The monocyte derived cells are administered at a dose of about 10⁶ to about 10⁹ cells/kg of body weight, particularly from about 10⁷ to about 10⁸ cells/kg of body weight.

The invention also relates to a vaccine containing as active substance monocyte derived cells presenting antigenic epitopes on their membranes according to the invention, or a ternary complex according to the invention, in association with a pharmaceutically acceptable vehicle.

The invention also relates to the use of monocyte derived cells according to the invention or of a ternary complex according to the invention, for the preparation of a medicament for treating cancer or infectious diseases.

The invention also relates to the method for the treatment or prevention of cancer comprising the use of monocyte derived cells according to the invention, or of a ternary complex according to the invention.

The invention also relates to a method for the treatment or prevention of viral or bacterial infections comprising the use of monocyte derived cells according to the invention, or of a ternary complex according to the invention.

The invention also relates to a method according to the invention, wherein the monocyte derived cells or ternary complex are administered systemically, subcutaneously, intravenously or in mucosal or lymphoid tissues.

The invention also relates to a method for inducing or increasing an immune response comprising the use of monocyte derived cells according to the invention, or the use of ternary complex according to the invention.

### EXAMPLES

Macrophages or monocyte derived cells of the following examples are prepared according to the methods described in the above-mentioned references.

1) SCID mice are inoculated subcutaneously with human tumor cells. They are also “humanized” by i.v. injection of about 10 million human total mononuclear cells. No immune response is seen and the tumor keeps growing to reach 1 square cm size. Mice are then injected intravenously either with

a) 10 μg of pan tumor carcinoma antigen,

b) with 0.1 mg of IgG anti-pan carcinoma Ag,

c) with both,

d) with 1 million activated autologous macrophages,

e) with both Ag plus Ab and macrophages.

The absence of objective response is seen in groups a), b), and c); partial transient response in groups d) and a tumor regression in group e) are documented by measurement of tumor size with calipets in two perpendicular directions.

2) Anti-HBV antibodies are mixed with HBs particles in a ratio 10/1 with about 10⁷ HBs/ml and incubated for 4 h at 37° C. in IMDM medium with about 10⁶/ml monocyte derived cells. The resulting monocyte derived cells of the invention induce the proliferation of cytotoxic T lymphocytes (autologous to the monocyte derived cells) specific for HBV (10⁶ monocyte derived cells resuspended per ml or RPMI medium added to 5.10⁵ T lymphocytes during 5 days, followed by measurement of tritiated thymidine incorporation).

3) The emergence of resistance to antibiotics in hospitals, even after triple therapies, requires the development of new vaccinal immunotherapies of infections. Monocyte derived cells obtained ex vivo are resuspended in culture medium in the presence a: of monoclonal or polyclonal antibodies specific for the resistant infectious agent (e.a. virus, staphylococci) or of anti-LPS antibodies for Gram negative bacteria, and b: in the presence of killed pathogens (pasteurisation) or membrane extracts.

A ternary complex is formed between the infectious antigen, the antibodies and the monocyte derived cells. These monocyte derived cells cultured at about 5.10⁶/ml in IMDM medium process the antibody-infectious antigen complex during 4 h incubation time at 37° C. The cells are centrifuged, resuspended in isotonic saline and injected locally or systemically to the patient to induce protective therapeutic immunity against the infectious agents that can be illustrated by the decrease in the infection titer and the presence of anti-infectious antibodies in peripheral blood. Its shown that in mice with lethal Gram(−) infections, the injection of macrophages having processed anti-LPS antibodies and heat killed bacteria results in immune protection of the animals.

The above-mentioned experiments have been performed with different subsets of monocyte derived cells expressing immunoglobulin Fc receptors, and in particular with macrophages.

1. Monocyte derived cells, in a purified form and substantially free of contaminants, presenting antigenic epitopes on their membranes after interiorization and processing of at least an antigen-antibody complex formed between an antigen and an antibody, under appropriate conditions, said epitopes corresponding to proteolytic degradation products of said antigen,
with said antibody being directed against a tumor or against an infectious agent, and
with said antigen being fragments of tumor or of infectious agent, including membranes or tumor apoptotic bodies, or purified tumor or infectious antigens or being recombinant tumor or infectious antigens.

2. Process for the preparation of monocyte derived cells presenting antigenic epitopes on their membranes after interiorization and processing of at least an antigen-antibody complex, formed between an antigen and an antibody, said process comprising the following steps:
a) preparation of the monocytes derived cells according to the following method:
1) recovery of blood derived mononuclear cells directly from blood apheresis or from blood bag collection, followed if necessary by centrifugation, to eliminate a substantial part of red blood cells granulocytes and platelets, and collection of peripheral blood leukocytes;
2) washing peripheral blood leukocytes obtained at the preceeding steps for instance by centrifugation (to remove 90% of platelets, red blood cells and debris) to obtain mononuclear cells;
3) resuspension of the total mononuclear cells obtained at the preceeding step in culture medium (RPMI or IMDM type) at 10⁶ to 2.10⁷ cells/ml, possibly completed by cytokines and/or autologous serum, and culture for 5 to 10 days at 37° C. under O₂/CO₂ atmosphere in hydrophobic gas permeable bags, to obtain monocyte derived cells and contaminating lymphocytes;
b) addition of antigens and antibodies to the monocyte derived cells obtained at the preceeding step to form a ternary complex between monocyte derived cells, an antigen and an antibody;
c) incubation of said ternary complex for a time and at a temperature sufficient to allow endocytosis into intracellular vacuoles of the monocyte derived cells and processing of the antigen-antibody complex, with said processing consisting of digestion of the antigen-antibody complex and association of the epitopes of the antigen resulting from the digestion with MHC molecules, to obtain monocyte derived cells presenting antigenic epitopes on their membranes.

3. Process according to claim 2, wherein
said antibody is directed against a tumor or against an infectious agent,
with said antigen is fragments of tumor or of infectious agent, including membranes or tumor apoptotic bodies, or purified tumor or infectious antigens or is a recombinant tumor or infectious antigen.

4. Process according to claim 2, wherein in the step of addition, said antigens and antibodies are in the form of a complex.

5. Process according to anyone of claims 1 to 4, comprising the additional following step:
d) centrifugation of the monocyte derived cells presenting antigenic epitopes on their membranes, washing and resuspension, for instance in isotonic medium, to obtain a suspension of the above defined monocyte derived cells.

6. Process according to claim 5, wherein the step of centrifugation is followed by
e) freezing at temperature below or equal to −80° C. aliquots of the above said suspension, with the addition of a cryopreservative.

7. Process according to claim 6, wherein the step of freezing is followed by
f) melting said above frozen aliquots at a temperature enabling to obtain a suspension of monocyte derived cells presenting antigenic epitopes on their membranes, for instance at 4° C., washing said suspension and resuspending it, for instance in an isotonic medium, to obtain a suspension of monocyte derived cells presenting antigenic epitopes on their membranes.

8. Process according to anyone of claims 2 to 7, wherein the antibodies are human or humanised IgG, IgA or preferably IgM (for induction of primary immune response) directed against tumor antigens or against infectious antigens (viral or bacterial).

9. Process according to anyone of claims 2 to 7, wherein the antigens are purified tumor, viral or bacterial antigens (polypeptides, glycopeptides, oligosacharides) or membrane fragments serving as complex antigens.

10. Process according to claims 8 or 9, wherein the antigens are pan tumor antigens present on different tumor types.

11. Ternary complex in a purified form, and substantially free of contaminants, between monocytes derived cells, an antigen and an antibody
with said antibody being directed against a tumor or against an infectious agent,
with said antigen being fragments of tumor or of infectious agent including membranes or tumor apoptotic bodies, or purified tumor or infectious antigens or being recombinant tumor or infectious antigens, and
with said antigen and said antibody being liable to form an antigen-antibody complex under appropriate conditions.

12. Process for the preparation of a ternary complex between monocyte derived cells, an antigen and an antibody according to claim 11, comprising the following steps:
1) recovery of blood derived mononuclear cells directly from blood apheresis or from blood bag collection, followed if necessary by centrifugation, to eliminate a substantial part of red blood cells granulocytes and platelets, and collection of peripheral blood leukocytes;
2) washing peripheral blood leukocytes obtained at the preceeding steps for instance by centrifugation (to remove 90% of platelets, red blood cells and debris) to obtain mononuclear cells;
3) resuspension of the total mononuclear cells obtained at the preceeding step in culture medium (RPMI or IMDM type) at 10⁶ to 2.10⁷ cells/ml, possibly completed by cytokines and/or autologous serum, and culture for 5 to 10 days at 37° C. under O₂/CO₂ atmosphere in hydrophobic gas permeable bags, to obtain monocyte derived cells and contaminating lymphocytes;
b) addition of antigens and antibodies to the monocyte derived cells obtained at the preceeding step to form of a ternary complex between monocyte derived cells, an antigen and an antibody.

13. Process according to claim 12, wherein said antigens and antibodies being either in the form of a complex or not.

14. Monocyte derived cells presenting antigenic epitopes on their membranes such as obtained according to the process of anyone of claims 2 to 10.

15. Ternary complex between monocyte derived cells, an antigen and an antibody such as obtained according to claims 13 or 14.

16. Pharmaceutical composition containing as active substance monocyte derived cells presenting antigenic epitopes on their membranes according to claims 1 or 14, in association with a pharmaceutically acceptable vehicle.

17. Pharmaceutical composition containing as active substance a ternary complex according to claim 15, in association with a pharmaceutically acceptable vehicle.

18. Pharmaceutical composition according to claims 16 or 17, in the form of sterile injectable preparations.

19. Vaccine containing as active substance monocyte derived cells presenting antigenic epitopes on their membranes according to claims 1 or 14, or a ternary complex according to claim 15, in association with a pharmaceutically acceptable vehicle.

20. Use of monocyte derived cells according to claims 1 or 14, or of a ternary complex according to claim 15, for the preparation of a medicament for treating cancer or infectious diseases.

21. Method for the treatment or prevention of cancer comprising the use of monocyte derived cells according to claims 1 or 14, or of a ternary complex according to claim 15.

22. Method for the treatment or prevention of viral or bacterial infections comprising the use of monocyte derived cells according to claims 1 or 14, or of a ternary complex according to claim 15.

23. Method according to claims 21 or 22, wherein the monocyte derived cells or ternary complex are administered systemically, subcutaneously, intravenously or in mucosal or lymphoid tissues.

24. Method for inducing or increasing an immune response comprising the use of monocyte derived cells according to claims 1 or 14, or the use of ternary complex according to claim 15.
